(19) United States
(12) Patent Application Publication
(10) Pub. No.: US 2025/0058008 A1
(43) Pub. Date: Feb. 20, 2025

(54) APPARATUS FOR DELIVERING A VOLATILE MATERIAL

(71) Applicant: The Procter & Gamble Company, Cincinnati, OH (US)

(72) Inventors: Rahul VYAS, Singapore (SG); Desmond NG, SINGAPORE (SG); Bhavesh RAJWANI, SINGAPORE (SG)

(21) Appl. No.: 18/802,105

(22) Filed: Aug. 13, 2024

Related U.S. Application Data
(60) Provisional application No. 63/519,409, filed on Aug. 14, 2023.

(51) Int. Cl. A61L 2/235; A61L 9/12

(52) U.S. Cl. CPC A61L 2/235; A61L 9/12; A61L 2209/131

(57) ABSTRACT

An apparatus for delivering a volatile material includes a delivery engine. The delivery engine includes a reservoir containing a volatile material; and a microporous membrane enclosing said reservoir. The microporous membrane has a volume average pore diameter of from 0.065 μm to 0.15 μm.

### TECHNICAL FIELD

The present disclosure relates to an apparatus for delivering a volatile material, particularly to an apparatus for delivering a volatile material to the environment within an enclosed space, such as a room or vehicle.

### BACKGROUND

It is generally known to use a device to evaporate a volatile material into a space, particularly a domestic space, in order to deliver a variety of benefits, such as air freshening or perfuming of the air. Non-energized systems, for example, systems that are not powered by electrical energy, are a popular way for the delivery of volatile materials into the atmosphere.

These systems can be classified into those that require human actuation, such as aerosols, and those which do not required human actuation, such as wick based systems and gels. The first type delivers the volatile materials on demand and the second type in a more continuous manner.

Variations on the second type of systems include membrane-based systems such as those disclosed in PCT patent publication WO 2010/120960A1. While such systems have enjoyed major commercial success, there remains the possibility of improvement. For example, a substantial amount of low-volatile materials may remain trapped on or within the membrane, and so it is desirable to improve the efficiency of release of volatile material (e.g. perfume). Furthermore, it can be difficult for consumers to accurately determine when existing membrane-based products have reached the end of their lifespan, which can result in consumer dissatisfaction.

There is a need for a membrane-based apparatus for delivering a volatile material which addresses at least some of the drawbacks associated with the prior art. There is also a need for a membrane-based apparatus having an improved perfume release profile.

### SUMMARY

The present disclosure addresses one or more of the drawbacks associated with the prior art. By providing an apparatus comprising a membrane having a volume average pore diameter of from 0.065 μm to 0.15 μm it has surprisingly been found that the following advantages are obtained.

First, the membrane enables a greater efficiency of use of perfume provided in the apparatus, with less volatile material left trapped on the membrane at the end of the product's lifespan. This improvement may also advantageously cause the membrane to have a substantially different appearance when wet with volatile material, as compared to its appearance when dry (whether before activation or at the end of the product's lifespan). This advantageously allows a consumer to easily determine whether or not the product has been activated properly, and also whether it needs replacing.

The membrane enables improved perfume release, especially during the middle and end portions of the product's lifespan. This benefit is surprisingly obtained whilst maintaining the same total product lifespan.

When using a membrane having a lower bulk density to prior art membranes, it is also possible to seal the membrane at a lower temperature, improving ease and cost of manufacture.

Therefore, the present disclosure provides the following.

1. An apparatus for delivering a volatile material comprising a delivery engine comprising:
a. a reservoir containing a volatile material; and
b. a microporous membrane enclosing said reservoir,
   wherein the microporous membrane has a volume average pore diameter of from 0.065 μm to 0.15 μm.

2. The apparatus of clause 1, wherein the delivery engine further comprises:
c. a rupturable substrate secured to said reservoir; and
d. a rupture element positioned adjacent to said rupturable substrate,
   wherein the microporous membrane encloses said rupturable substrate and said rupture element.

3. The apparatus of clause 1 or 2, wherein the microporous membrane has a surface area of from 2 cm² to 100 cm²,
optionally from 2 cm² to 35 cm².

4. The apparatus of any one of the preceding clauses, wherein the microporous membrane has a porosity of from 45 to 70%,
optionally from 45 to 60%.

5. The apparatus of any one of the preceding clauses, wherein the microporous membrane has a total pore volume of from 0.6 to 2 cm³/g,
optionally from 0.65 to 1.6 cm³/g,
more optionally from 0.7 to 1.5 cm³/g.

6. The apparatus of any one of the preceding clauses, wherein the microporous membrane has a bulk density of from 0.3 to 0.8 g/cm³,
optionally from 0.35 to 0.75 g/cm³,
more optionally from 0.4 to 0.7 g/cm³.

7. The apparatus of any one of the preceding clauses, wherein the microporous membrane has a thickness of from 0.2 to 0.4 mm,
optionally from 0.22 to 0.37 mm,
more optionally from 0.25 to 0.35 mm.

8. The apparatus of any one of the preceding clauses, wherein the microporous membrane has:
a porosity of from 45 to 60%;
a total pore volume of from 0.65 to 1.5 cm³/g; and
a bulk density of from 0.35 to 0.75 g/cm³.

9. The apparatus of any one of the preceding clauses, wherein the microporous membrane is not laminated.

10. The apparatus of any one of the preceding clauses, wherein the microporous membrane comprises polyethylene,
optionally wherein the polyethylene is ultra-high molecular weight polyethylene (UHMWPE).

11. The apparatus of any one of the preceding clauses, wherein the microporous membrane:
comprises polyethylene;
has a thickness of from 0.2 to 0.4 mm; and
is not laminated.

12. The apparatus of any one of the preceding clauses, wherein the reservoir contains a volatile material, which volatile material is liquid at 25° C.,
optionally wherein the volatile material has a vapor pressure of at least 8 Pa at 25° C.,
more optionally wherein the volatile material has a vapor pressure of at least 30 Pa at 25° C.

13. The apparatus of any one of the preceding clauses, wherein:
the delivery engine further comprises:
c. a rupturable substrate secured to said reservoir; and
d. a rupture element positioned adjacent to said rupturable substrate,
the microporous membrane encloses said rupturable substrate and said rupture element;
the reservoir contains a volatile material;
the apparatus is configured such that activation of the rupture element allows contact between the volatile material and the microporous membrane; and
the apparatus is configured such that after activation of the rupture element, the apparatus releases at least 80 wt. % of the volatile material within a time period of 8 weeks at a temperature of 25° C.

14. The apparatus of any one of the preceding clauses, wherein the microporous membrane has a first visible state when dry, and a second visible state when wetted with volatile material, and where a CIE2000 Delta E value between the first visible state and second visible state is greater than or equal to 5.

15. The apparatus of any one of the preceding clauses, wherein the microporous membrane has a first visible state when dry, and a second visible state when wetted with volatile material, and where a difference between a luminous transmittance value for the first visible state and a luminous transmittance value for the second visible state, as measured by ISO 13468-2:2021, is greater than or equal to 25%.

### BRIEF DESCRIPTION OF THE DRAWINGS

FIG. 1 shows a perspective view of one example of an apparatus in accordance with the present disclosure.

FIG. 2 shows an exploded, perspective view of one example of a delivery engine in accordance with the present disclosure.

FIG. 3 is a front perspective view of a volatile composition dispenser according to an example.

FIG. 4 is a rear perspective view of the volatile composition dispenser shown in FIG. 3.

### DETAILED DESCRIPTION

The present disclosure provides an apparatus for delivering a volatile material comprising a delivery engine comprising:
a. a reservoir containing a volatile material; and
b. a microporous membrane enclosing said reservoir,
   wherein the microporous membrane has a volume average pore diameter of from 0.065 μm to 0.15 μm.

As used herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and examples of the present disclosure. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure. When used herein, the term “substantially identical” is intended to refer to a dimension that is essentially identical, but for variations resulting from manufacturing tolerances. For example, the term may mean that a dimension varies by less than 5%, such as less than 2%, such as less than 1%, such as less than 0.5%, such as less than 0.05%, such as the dimension is essentially uniform.

Preferably, the present disclosure relates to a non-energized apparatus for the delivery of a volatile material to the atmosphere in a continuous, non-energized manner. “Non-energized” means that the apparatus is passive does not require to be powered by a source of external energy. In particular, the apparatus does not need to be powered by a source of heat, gas, or electrical current, and the volatile material is not delivered by aerosol means. Further, as used in this specification and the appended claims, the singular forms “a”, “an”, and “the” include plural references unless the content clearly dictates otherwise. Thus, for example, “a volatile material” may include more than one volatile material.

The apparatus of the present disclosure delivers a volatile material in a substantially continuous manner when the apparatus is in a resting position (i.e. the apparatus is not being moved). The emission level of volatile materials may exhibit a uniform intensity until substantially all the volatile materials are exhausted. The continuous emission of the volatile materials can be of any suitable length, including but not limited to, up to: 20 days, 30 days, 60 days, 90 days, shorter or longer periods, or any period between 30 to 90 days, such as about 8 weeks (56 days).

The apparatus of the present disclosure is suitable for purposes of providing fragrances, air fresheners, deodorizers, odor eliminators, malodor counteractants, insecticides, insect repellents, medicinal substances, disinfectants, sanitizers, mood enhancers, and aromatherapy aids, or for any other purpose using a volatile material that acts to condition, modify, or otherwise change the atmosphere or the environment. For purposes of illustrating the present disclosure in detail, but without intending to limit the scope of the present disclosure, the apparatus of the present disclosure will be described in an air freshening system for delivering liquid containing perfume raw materials.

The present disclosure is based on the surprising finding that an apparatus comprising a microporous membrane having a volume average pore diameter of from 0.065 μm to 0.15 μm provides several advantages as discussed herein. The apparatus may be of the type discussed in, for example, U.S. Pat. No. 8,740,110 (U.S. Pat. No. 8,740,110), or United States of America Patent Application Publication No. 20220047754 (US 2022/0047754), both of which are incorporated herein by reference. However, a person skilled in the art will appreciate that the surprising advantages associated with the apparatus of the present disclosure may be obtained using other apparatus, and the present disclosure is not limited to apparatus of the type disclosed in U.S. Pat. No. 8,740,110 or US 2022/0047754.

As discussed herein, the present disclosure provides an apparatus for delivering a volatile material. The apparatus comprises a delivery engine, which is to be understood to mean a part of the apparatus that is capable of delivering a volatile material to the surrounding atmosphere.

The delivery engine comprises:
a. a reservoir containing a volatile material; and
b. a microporous membrane enclosing said reservoir, the microporous membrane having a volume average pore diameter of from 0.065 μm to 0.15 μm.

The reservoir contains a volatile material. The microporous membrane encloses the reservoir such that volatile material is unable to escape from the delivery engine without passing through the microporous membrane. Since the microporous membrane prevents the passage of liquid, the volatile material is only able to escape the delivery engine by evaporating through, or from, the microporous membrane.

### Microporous Membrane

The microporous membrane is vapor permeable and capable of wicking liquid, yet prevents free flow of liquid out of the membrane. The microporous membrane has a volume average pore diameter of from 0.065 μm to 0.15 μm. The use of a microporous membrane having such a pore size provides a number of advantages as discussed herein and demonstrated in the below Examples.

Without being bound by theory, it is believed that microporous membranes having a volume average pore diameter of less than 0.065 μm will provide inferior perfume release, and will not provide other advantages that are obtained by the current apparatus of the present disclosure. It is also believed that microporous membranes having higher volume average pore diameters may suffer from leaking and/or sweating.

The microporous membrane is vapor permeable and capable of wicking liquid, yet prevents free flow of liquid out of the membrane. The microporous membrane may have limited selectivity such that it prevents the passage of fewer perfume materials compares to traditional membranes. Membranes that are selective, such as traditional polyethylenes, may inhibit high molecular weight volatile materials and materials with low solubility in polyethylene from diffusing through. This may limit perfume formulations, for example in the field of air fresheners where it is typically desired to use formulations having a wide variety of volatile materials having different volatilities (e.g. top notes, middle notes and bottom notes). For example, some membranes may preclude the diffusion of alcohols, such as linalool and dihydromyrcenol which are widely used in perfume applications.

The microporous membrane has a volume average pore diameter of from 0.065 μm to 0.15 μm. The microporous membrane may have a volume average pore diameter of from 0.07 to 0.12 μm, such as from 0.07 to 0.11 μm, or 0.08 to 0.1 μm.

Typically, the microporous membrane has a pore size distribution such that at least 50%, such as at least 60%, such as at least 70%, such as at least 80% or such as at least 90% of the pores of the microporous membrane have a pore diameter of from 0.065 μm to 0.15 μm.

The microporous membrane may comprise (e.g. be formed from) polyethylene, such as ultra-high molecular weight polyethylene (UHMWPE), though other length polyethylene chains may also be used. As used herein, UHMWPE refers to polyethylene having a molecular mass of from about 3.5 million to 7.5 million amu.

The microporous membrane may have a thickness in the z-direction, of about 0.01 mm to about 1 mm, alternatively between about 0.2 mm to about 0.4 mm, from about 0.22 to about 0.37 mm, e.g. from about 0.25 to about 0.35 mm.

It is herein explicitly contemplated that any end point of any range defined in relation to a variable disclosed herein may be combined with any other end point from any other range defined in relation to the same variable. Thus, for the thickness ranges discussed above, the following ranges are also explicitly contemplated, and it is to be understood that the same principle may be applied to ranges disclosed herein for any other variable:
from 0.01 to 0.2 mm, from 0.01 to 0.22 mm, from 0.01 to 0.25 mm, from 0.01 to 0.35 mm, from 0.01 to 0.37 mm, from 0.01 to 0.4 mm, from 0.01 to 1 mm;
from 0.2 to 0.22 mm, from 0.2 to 0.25 mm, from 0.2 to 0.35 mm, from 0.2 to 0.37 mm, from 0.2 to 0.4 mm, from 0.2 to 1 mm;
from 0.22 to 0.25 mm, from 0.22 to 0.35 mm, from 0.22 to 0.37 mm, from 0.22 to 0.4 mm, from 0.22 to 1 mm;
from 0.25 to 0.35 mm, from 0.25 to 0.37 mm, from 0.25 to 0.4 mm, from 0.25 to 1 mm;
from 0.35 to 0.37 mm, from 0.35 to 0.4 mm, from 0.35 to 1 mm;
from 0.37 to 0.4 mm, from 0.37 to 1 mm; and
from 0.4 to 1 mm.

The microporous membrane may be formed from a single piece, or single sheet, of material. In other words, the microporous membrane may not be laminated. Thus, the microporous membrane may be formed from a single sheet of polyethylene having a thickness as described above.

Those of ordinary skill in the art will appreciate that the surface area of the microporous membrane can vary depending on the user preferred size of the delivery engine. In some examples, the (evaporative) surface area of the microporous membrane may be about 2 cm² to about 100 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm².

The microporous membrane may have any appropriate porosity. For example, the microporous membrane may have a porosity of from 45% to 70%, on a volume basis, such as from 45% to 65%. In certain examples of the present disclosure, the porosity may be from 50 to 70%, such as 55 to 65%.

The microporous membrane may have any appropriate total pore volume, such as from 0.6 to 2 cm³/g. Typically, the total pore volume may be from 0.65 to 1.6 cm³/g, such as 0.7 to 1.5 cm³/g. In certain examples of the present disclosure, the total pore volume may be from 0.8 to 1.4 cm³/g.

The microporous membrane may have any appropriate bulk density, such as from 0.3 to 0.8 g/cm³. Typically, the bulk density may be from 0.35 to 0.75 g/cm³, such as from 0.4 to 0.7 g/cm³. In certain examples of the present disclosure, the bulk density may be from 0.4 to 0.6 g/cm³.

Suitable microporous membranes for the present disclosure include microporous polyethylene membranes having the properties described herein, available from Microporous, LLC.

The microporous membrane may comprise any suitable filler and plasticizer known in the art. Fillers may include finely divided silica, clays, zeolites, carbonates, charcoals, and mixtures thereof. In one example, the microporous membrane may be filled with about 30% to about 80%, by total weight, of silica.

In one aspect of the present disclosure, the microporous membrane may include a dye that is sensitive to the amount of volatile material it is in contact with to indicate end-of-life. Alternatively, the microporous membrane may change to transparent when in contact with a fragrance or volatile material to indicate diffusion is occurring. Other means for indicating end-of-life that are known in the art are contemplated for the present disclosure.

The membranes described herein may advantageously provide a clear visual change when wetted with volatile material, and when dry (whether before use or at end of life). Such visual changes may be more detectible when the membrane does not comprise a white pigment (e.g. TiO₂). Therefore, the microporous membrane may comprise less than 5 wt. % of a white pigment, such as less than 1 wt. % of a white pigment, less than 0.1 wt. % of a white pigment, or less than 0.01 wt. % of a white pigment. The microporous membrane may be free from a white pigment.

The visual change when the membrane is wetted as compared to dry may be more noticeable when the microporous membrane comprises a coloured dye/pigment or a black dye/pigment. Therefore, the microporous membrane may comprise a coloured or black dye/pigment, such as activated charcoal. Such a coloured or black pigment/dye (e.g. activated charcoal) may be present in any suitable amount, such as from 0.1 to 5 wt. %, e.g. 0.3 to 1 wt. %.

### Volatile Material

The term “volatile material” as used herein, refers to a material that is vaporizable at room temperature and atmospheric pressure without the need of an energy source. The volatile material may be a composition comprised entirely of a single volatile material. The volatile material may also be a composition comprised entirely of a volatile material mixture (i.e. the mixture has more than one volatile component). Further, it is not necessary for all of the component materials of the composition to be volatile. Any suitable volatile material in any amount or form, including a liquid or emulsion, may be used.

Liquid suitable for use herein may, thus, also have non-volatile components, such as carrier materials (e.g., water, solvents, etc). It should also be understood that when the liquid is described herein as being “delivered”, “emitted”, or “released,” this refers to the volatilization of the volatile component thereof, and does not require that the non-volatile components thereof be emitted.

The volatile material can be in the form of perfume oil. Most conventional fragrance materials are volatile essential oils. The volatile material can be a volatile organic compound commonly available from perfumery suppliers. Furthermore, the volatile material can be synthetically or naturally formed materials. Examples include, but are not limited to: oil of bergamot, bitter orange, lemon, mandarin, caraway, cedar leaf, clove leaf, cedar wood, geranium, lavender, orange, origanum, petitgrain, white cedar, patchouli, neroili, rose absolute, and the like. In the case of air freshener or fragrances, the different volatile materials can be similar, related, complementary, or contrasting.

The volatile material may also originate in the form of a crystalline solid, which has the ability to sublime into the vapor phase at ambient temperatures or be used to fragrance a liquid. Any suitable crystalline solid in any suitable amount or form may be used. For example, suitable crystalline solids include but are not limited to: vanillin, ethyl vanillin, coumarin, tonalid, calone, heliotropene, musk xylol, cedrol, musk ketone benzohenone, raspberry ketone, methyl naphthyl ketone beta, phenyl ethyl salicylate, veltol, maltol, maple lactone, proeugenol acetate, evemyl, and the like.

Nevertheless, it may be desirable for the volatile material to be in the form of a liquid at 25° C. As explained herein, the microporous membranes used in the apparatus of the present disclosure may have advantageously increased visual appearance changes when wetted with volatile material. This advantageously, quickly and clearly confirms to a user that the volatile material is in contact with the microporous membrane (confirming that an apparatus has been activated properly, if activation is required). The appearance change also clearly confirms that an apparatus has reached the end of its life, since the membrane appearance will revert back to the dry appearance.

Thus, the microporous membrane may have a first visible state when dry, and a second visible state when wetted with volatile material, where the first and second visible states have a different appearance. For example, a CIE2000 Delta E value for difference in sRGB for the first visible state and second visible state may be greater than or equal to 5, such as greater than or equal to 8, greater than or equal to 10, or greater than or equal to 12. As used herein, CIE2000 Delta E refers to Delta E (ΔE*) as calculated by the CIE2000 formula published by the International Commission on Illumination (CIE). Alternatively, or in addition, a difference between a luminous transmittance value for the first visible state and a luminous transmittance value for the second visible state, as measured by ISO 13468-2:2021, may be greater than or equal to 25%, such as greater than or equal to 30%, greater than or equal to 35%, greater than or equal to 40%, or greater than or equal to 45%.

The volatile material may have a combined vapour pressure of at least 8 Pa at 25° C., such as at least 30 Pa at 25° C.

It may not be desirable for volatile materials to be closely similar if different volatile materials are being used in an attempt to avoid the problem of emission habituation. Otherwise, the people experiencing the emissions may not notice that a different material is being emitted. The different emissions can be provided using a plurality of delivery systems each providing a different volatile material (such as, musk, floral, fruit emissions, etc). The different emissions can be related to each other by a common theme, or in some other manner. An example of emissions that are different, but complementary might be a cinnamon emission and an apple emission.

In addition to the volatile material of the present disclosure, the delivery engine may include any known malodor composition to neutralize odors. Suitable malodor compositions include cyclodextrin, reactive aldehydes and ionones.

While not wishing to be bound by theory, the continuous delivery of a volatile material may be a function of various factors including membrane pore size; membrane surface area; the physical properties of a volatile material, such as molecular weight and saturation vapor pressure (“VP”); and the viscosity and/or surface tension of the composition containing the volatile material.

The composition may be formulated such that the composition comprises a volatile material mixture comprising about 10% to about 100%, by total weight, of volatile materials that each having a VP at 25° C. of less than about 0.01 torr; alternatively about 40% to about 100%, by total weight, of volatile materials each having a VP at 25° C. of less than about 0.1 torr; alternatively about 50% to about 100%, by total weight, of volatile materials each having a VP at 25° C. of less than about 0.1 torr; alternatively about 90% to about 100%, by total weight, of volatile materials each having a VP at 25° C. of less than about 0.3 torr. In one example, the volatile material mixture may include 0% to about 15%, by total weight, of volatile materials each having a VP at 25° C. of about 0.004 torr to about 0.035 torr; and 0% to about 25%, by total weight, of volatile materials each having a VP at 25° C. of about 0.1 torr to about 0.325 torr; and about 65% to about 100%, by total weight, of volatile materials each having a VP at 25° C. of about 0.035 torr to about 0.1 torr. One source for obtaining the saturation vapor pressure of a volatile material is EPI Suite™, version 4.0, available from U.S. Environmental Protection Agency.

Two exemplary compositions comprising a volatile material mixture having volatile materials of varying VPs are set forth below in Tables 1 and 2. These compositions are shown by way of illustration and are not intended to be in any way limiting of the present disclosure.

TABLE 1

| Wt % | Low VP (torr) | High VP (torr) |
|---|---|---|
| 27.71 | 0.175 | 0.325 |
| 20.78 | 0.0875 | 0.1125 |
| 13.86 | 0.0625 | 0.0875 |
| 8.66 | 0.0375 | 0.0625 |
| 8.66 | 0.0175 | 0.0325 |
| 6.93 | 0.00875 | 0.01125 |
| 6.93 | 0.00625 | 0.00875 |
| 3.18 | 0.00375 | 0.00625 |
| 1.27 | 0.00175 | 0.00325 |
| 0.95 | 0.000875 | 0.001125 |
| 0.64 | 0.000625 | 0.000875 |
| 0.32 | 0.000375 | 0.000625 |
| 0.09 | 0.000175 | 0.000325 |

TABLE 2

| Wt % | Low VP (torr) | High VP (torr) |
|---|---|---|
| 33.38 | 0.175 | 0.325 |
| 25.75 | 0.0875 | 0.1126 |
| 19.07 | 0.0625 | 0.0875 |
| 13.86 | 0.0375 | 0.0625 |
| 4.00 | 0.0175 | 0.0325 |
| 1.50 | 0.00875 | 0.01125 |
| 0.50 | 0.00625 | 0.00875 |
| 0.72 | 0.00375 | 0.00625 |
| 0.55 | 0.00175 | 0.00325 |
| 0.27 | 0.000875 | 0.001125 |
| 0.20 | 0.000625 | 0.000875 |
| 0.13 | 0.000375 | 0.000625 |
| 0.07 | 0.000175 | 0.000325 |

The viscosity of a volatile material may control how and when a volatile material is delivered to the microporous membrane. For example, less viscous compositions may flow faster than the more viscous volatile materials. Thus, the membrane may be first wetted with the less viscous materials. To help prevent liquid from seeping through the microporous membrane, volatile materials may have viscosities less than about 23 cP and surface tension less than about 33 mN/m.

In one example, the composition containing a volatile material may have a viscosity of about 1.0 cP to less than about 25 cP, alternatively about 1.0 cP to less than about 23, alternatively about 1.0 cP to less than about 15 cP.

The composition containing a volatile material may be designed such that the composition may include a surface tension of about 19 mN/m to less than about 33 mN/m, alternatively about 19 mN/m to less than about 30 mN/m, alternatively about 19 mN/m to less than about 27 mN/m.

The delivery engine may further comprise:
c. a rupturable substrate secured to said reservoir; and
d. a rupture element positioned adjacent to said rupturable substrate,
   wherein the microporous membrane encloses said rupturable substrate and said rupture element.

The rupturable substrate serves to prevent contact between the volatile material and microporous membrane before the apparatus is desired to be used. The rupturable substrate may be ruptured by actuating a rupture element, and such rupturing of the rupturable substrate will allow volatile material to flow through the rupturable substrate and contact the microporous membrane. The configuration of the rupturable substrate and rupture element is described in more detail hereinbelow.

Purely by way of illustration, the apparatus of the present disclosure is described in more detail below with reference to the Figures.

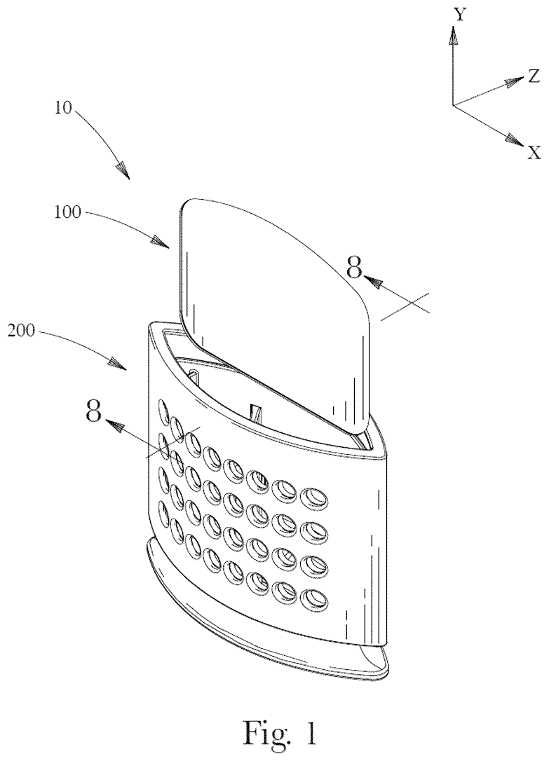

FIG. 1 shows a first example of an apparatus 10. The apparatus 10 corresponds to those described in U.S. Pat. No. 8,740,110, and includes a delivery engine 100 and a housing 200, and has a cross section 8. The present disclosure provides such apparatus comprising a microporous membrane having a volume average pore diameter of from 0.065 μm to 0.15 μm.

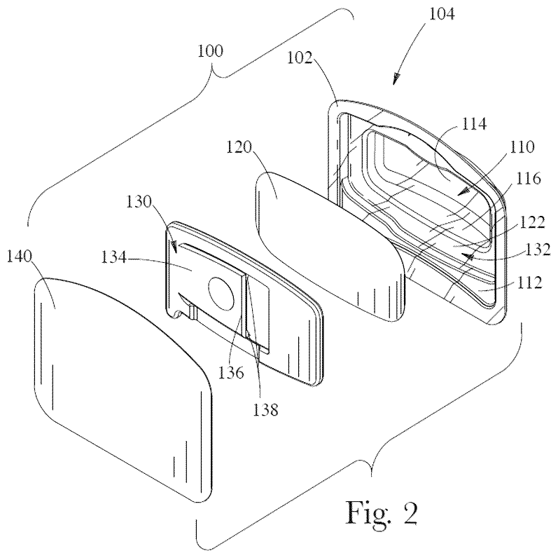

FIG. 2 shows the delivery engine 100 of FIG. 1, which comprises a width, length and depth along an x-axis, y-axis, and z-axis, respectively. The width, length, and depth may be such that the delivery engine 100 is considered compact and/or portable. By “compact” or “portable”, it is meant that the delivery engine 100 can be conveniently and comfortably carried in a pocket, purse, or the like. The delivery engine 100 can be constructed as a disposable, single-use item or one that it is replenished with a volatile material.

The delivery engine 100 may include a lip 102 that defines the outer perimeter of the delivery engine 100 and may circumference a reservoir 110 containing a volatile material as well as a collection basin 112. The delivery engine 100 may also include a rupturable substrate 120 secured to the reservoir 110; a rupture element 130 positioned adjacent to the rupturable substrate 120; and a microporous membrane 140 secured to the lip 102 and enclosing the rupturable substrate 120, reservoir 110, and collection basin 112.

The body 104 of the delivery engine 100 can be thermoformed, injection molded, or blow molded with any known material. In some examples, the body 104 includes all structural aspects of the delivery engine 100 minus the rupturable substrate 120, the rupture element 130, and microporous membrane 140. In other examples, the body 104 includes the rupture element 130. The body 104 may be made of a multi-layer material which may include a barrier layer to prevent evaporation of a volatile component and at least one outer layer that allows a rupturable substrate 120 to be heat-sealed to the body 104. A suitable sealant layer would include a layer of polyethylene or polypropylene or any suitable polyolefin sealant that allows for a leak proof seal of the reservoir 110. Suitable materials to form the body 104 of the delivery engine 100 include plastics, such as Pentaplast Pentaform® 2101 available from Klockner. In some examples, the material is colored or non-colored see-through plastic. The see-through material permits observation of the liquid and end-of life.

The delivery engine 100 may comprise a reservoir 110 for holding a volatile material. The reservoir 110 includes a width, length, and depth along the x-axis, y-axis, and z-axis, respectively. The reservoir 110 may be elongate in that its width to length ratio is about 2:1 to about 4:1, alternatively about 1.5:1 to about 2.5:1. The reservoir 110 may have a width of about 45 mm to about 55 mm, alternatively about 51 mm; a length of about 15 mm to about 30 mm to about, alternatively about 23 mm; a depth of about 5 mm to about 15 mm, alternatively about 11 mm. The dimensions of the reservoir 110 may be such that it holds about 2 ml to about 50 ml of liquid containing a volatile material.

Alternatively, the reservoir 110 may hold about 2 ml to about 30 ml, alternatively about 2 ml to about 10 ml, alternatively about 2 ml to about 8 ml, alternatively about 4 ml to about 6 ml, alternatively about 2 ml, alternatively about 6 ml of liquid containing a volatile material.

The reservoir 110 may include a bottom 114 and a single opening 116. The reservoir 110 may also have a ridge 122 circumferencing the single opening 116 or the upper edge of the reservoir 110. This ridge 122 may provide a generally flat surface upon which a rupturable substrate 120 may be secured. The ridge 122 allows the secured area of the rupturable substrate 120 to be located away from the inner walls of the reservoir 110 where the volatile material would be held.

It is contemplated that the delivery engine 100 of the present disclosure may comprise two or more reservoirs (not shown) which can be filled with the same or different volatile materials. The reservoirs may have any configuration that contacts the microporous membrane 140 upon rupture. For example, the reservoirs may be opposedly connected for use in a flippable device. In such a device, the microporous membrane 140 is fluidly connected between the reservoirs.

The delivery engine 100 may include a rupturable substrate 120. The rupturable substrate 120 may be configured in any manner that prevents the volatile material in the reservoir 110 from contacting the microporous membrane 140 prior to activating or rupturing the delivery engine 100. In one example, the rupturable substrate 120 may enclose the reservoir, prior to activation, by extending across the single opening 116 securing to the ridge 122 of the reservoir 110. The rupturable substrate 120 may be secured by a layer of adhesives, heat and/or pressure sealing, ultrasonic bonding, crimping, and the like or a combination thereof.

The rupturable substrate 120 can be made of any material that ruptures with applied force, with or without the presence of an element to aid in such rupture. Because the rupturable substrate 120 is intended to contain a volatile material while in storage, it may be made from a layer of barrier material that prevents evaporation of the volatile material prior to its intended use and a layer of heat-sealable layer. Such materials may be impermeable to vapors and liquids. Suitable barrier materials for the rupturable substrate 120 include a flexible film, such as a polymeric film, a flexible foil, or a composite material such as foil/polymeric film laminate. Suitable flexible foils include a metal foil such as a foil comprised of a nitrocellulose protective lacquer, a 20 micron aluminum foil, a polyurethane primer, and 15 g/m2 polyethylene coating (Lidfoil 118-0092), available from Alcan Packaging. Suitable polymeric films include polyethylene terephtalate (PET) films, acrylonitrile copolymer barrier films such as those sold under the tradename Barex® by INOES, ethylene vinyl alcohol, and combinations thereof. It is also contemplated that coated barrier films may be utilized as a rupturable substrate 120. Such coated barrier films include metalized PET, metalized polypropylene, silica or alumina coated film may be used. Any barrier material, whether coated or uncoated, may be used alone and or in combination with other barrier materials.

The rupturable substrate 120 may be breached to release a volatile material by actuating a rupture element 130. The rupture element 130 can be injection, compression, or pressure molded using a polyolefin, such as polyethylene or polypropylene; polyester; or other plastics as known to be suitable for molding. The rupture element 130 could also be made by thermoforming with a discrete cutting step to remove parts not wanted. The rupture element 130 may be positioned in a space 132 formed in the delivery engine body 104 that is adjacent to the rupturable substrate 120 and subjacent a microporous membrane 140. The space 132 may be configured such that the rupture element 132 is nested within the space 132 and enclosed by a microporous membrane 140, thus requiring no other means to hold the rupture element 132 in the delivery engine 100. In one example, the rupture element 130 is positioned between and in contact with said rupturable substrate 120 and said microporous membrane 140. A rupture element 130 that is directly adjacent to the microporous membrane 140 may facilitate wetting of the microporous membrane 140. More specifically, liquid may wick between rupture element 130 and the microporous membrane 140 allowing for maintenance of a larger wetted surface area of the microporous membrane 140.

The rupture element 130 may be configured in any manner such that a user can manually actuate the rupture element 130 and breach the rupturable substrate 120 with relative ease. In one example, a user may actuate the rupture element 130 by manually compressing it. In other examples, the rupture element 130 may breach the rupturable substrate 120 through contact with an element provided in a delivery engine housing that engages and compresses the rupture element 130. Suitable compression forces to breach the rupturable substrate 120 with a rupture element 130 may be less than about 25N, alternatively, less than about 20N, alternatively less than about 15N, alternatively less than about ION, alternatively less than about 5N, alternatively from about 1N to about 15N, alternatively, from about 1N, to about ION, alternatively, from about 1N to about 5N.

The compression force can be measured using an electromechanical testing system, QTest Elite 10, available from MTS, along with a modified UL 283 finger probe made of polyamide. The UL 283 finger probe is described in Standard for Air Fresheners and Deodorizers, UL Standard 283, FIG. 10.1 (UL Mar. 31, 2004). As described in UL 283, FIG. 10.1, the radius of the finger tip is 3.5 mm; height of the finger tip is 5 mm; depth of the finger tip is 5.8 mm. However, unlike the finger probe described in the aforementioned text, the modified UL 283 finger probe does not include any articulating joints. Instead, it is in a fixed position that is perpendicular to the rupture element 130 when testing is conducted. The testing occurs at ambient temperatures (23±2° C.). The perimeter of a delivery engine 100 is rested on a support fixture, without directly contacting or directly securing the rupture element 130 to the support fixture. The crosshead speed of the electromechanical testing system is set at 30 mm/min. The modified UL 283 finger probe is moved towards the rupture element 130 to contact a region where displacement is desired for rupturing a rupturable substrate 120. Where a flange 134 such as the one described herein is utilized, the desired region of displacement is the mid-point of the flange 134. The mid-point is the point that is half way between the proximal end and distal end 136. For example, where a flange 134 is 2 cm from proximal end to distal end 136, the mid-point is located at 1 cm. The machine is run until the rupture element 130 is displaced by 6 mm. Zero displacement is defined as the point at which 0.1N of force (i.e. preload) is applied. The load at the first peak where the rupturable substrate 120 is broken is recorded as the force to rupture. Those of ordinary skill in the art will appreciate that compression forces will vary depending on the physical properties and placement of the microporous membrane 140, rupture element 130, and rupturable substrate 120 in a delivery engine 100.

There are numerous examples of the rupture element 130 described herein, all of which are intended to be non-limiting examples. FIG. 2 shows one non-limiting example of the rupture element 130. In this example, the rupture element 130 includes a flange 134 hinged to the rupture element 130. The flange 134 may be injection molded and may include a distal end 136. The distal end 136 may include one or more piercing elements 138 located in the z-direction or towards the rupturable substrate 120. In one example, the distal end 136 may include two spaced apart piercing elements 138 in the z-direction. In an alternate example, the distal end 136 may form a single point (not shown) along the x-y plane. A user may manually compress or press downward in the z-direction on the flange 134 such that the rupturable substrate 120 is breached and a volatile material is released to the microporous membrane 140.

It is contemplated that the rupture element 130 may include more than one flange 134 where additional points of rupture are desired. For example, the rupture element 130 may include a first compressible flange and a second compressible flange opposedly hinged to said rupture element (not shown).

The delivery engine 100 shown in FIG. 2 includes a microporous membrane 140 as described hereinabove.

When used with an apparatus according to FIG. 1, the microporous membrane 140 may be secured to the lip 102 of the delivery engine 100 in the same manner as the rupturable substrate 120 is secured to the ridge 122 of the reservoir 110. The microporous membrane 140 encloses the reservoir 110, rupturable substrate 120, rupture element 130, and collection basin 112. In this way, the rupturable substrate 120 may be breached by compressing the microporous membrane 140 and the rupture element 130. Once breached, the volatile material flows out of the reservoir 110, contacts the microporous membrane 140, and is delivered to the atmosphere. Because the microporous membrane 140 is shielded from the volatile material until the rupturable substrate 120 is breached, the fragrance intensity may build slowly from zero to its equilibrium rate of release when the microporous membrane 140 is fully wetted.

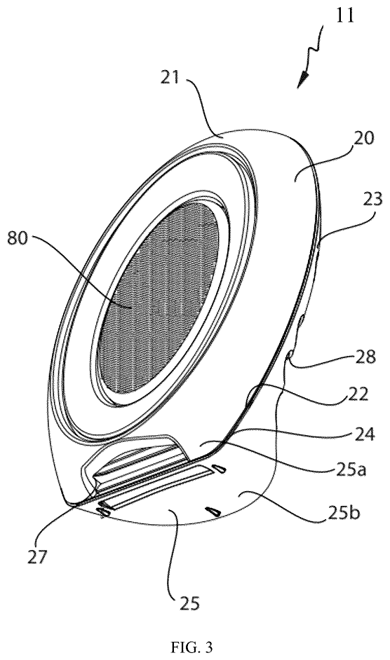

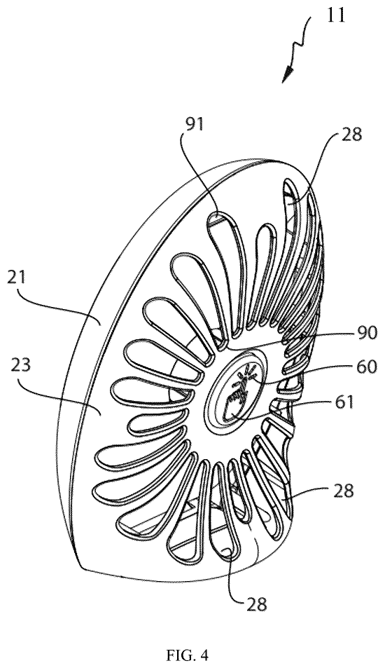

FIG. 3 shows a front perspective view of an example of an apparatus 11, corresponding to those described in US 2022/0047754, while FIG. 4 shows a rear perspective view of this example. The present disclosure provides such apparatus comprising a microporous membrane having a volume average pore diameter of from 0.065 μm to 0.15 μm. The apparatus 11 depicted comprises a housing 20 having a first wall 21 which opposes a second wall 23. The constituents of the housing 20, including the first and second walls may be made from plastic, bamboo, wood, glass, shell, pulp, metals, or metalloids. It is also foreseeable in certain examples that the selected material of the walls may be recyclable or even made from recyclable materials. Any of the components of the housing which include the first and second walls as well as the button and button channel may be molded via thermal means, injection, or by blowing. These first and second walls are joined to each other along their respective peripheries 22, 24. These walls may be joined to one another by various mechanisms including snap fit connectors, glue, or one or more latches that mechanically attaches one of the walls to the other. The first wall 21 and second wall 23 may be individually convex and may even be so convex as to form two hemispherical walls separately and a sphere shaped apparatus when joined to one another. In the depicted example, however, the first wall 21 and second wall 23 are each curvedly contoured in an elliptical shell form. Therefore, in this instance, they form an elliptical shaped disc housing and apparatus. One might say that the first and second walls of this example are shell shaped. The first wall 21 includes a window 80 and a primary aperture 27. The apparatus 11 comprises a base portion 25 made up of one or both of the first wall 22 (25a) and the second wall 24 (25b). In FIG. 3, the primary aperture 27 is disposed near a base portion 25a of the first wall. The primary aperture may vary in size but may have an area from about 30 mm², 40 mm², 50 mm², 60 mm², 70 mm², 80 mm², 90 mm² or even 100 mm² to about 120 mm², 130 mm², 140 mm², 150 mm², 160 mm², 170 mm², or 180 mm². In this example, the primary aperture 27 is about 110 mm².

The window 80 is useful for providing a user with the ability to be able to visually gauge the volume of the volatile composition within the receptacle of the cartridge. This window 80 easily accommodates a rear or bottom surface of a cartridge in most cases it will be transparent or translucent to facilitate in the viewing of the volume. This window 80 may take on various shapes. In this example, it is oval shaped but it may be rectangular, circular, triangular, or other asymmetric shapes that allow a user sufficient sight of the receptacle. The window 80 may also be of variable sizing. In an oval or elliptical configuration of the apparatus, the length may range from about 3 cm, 3.5 cm, 4 cm, 4.5 cm, or 5 cm to about 7 cm, 7.5 cm, 8 cm, 8.5 cm or 9 cm, while the width ranges from about 3 cm, 3.5 cm, or 4 cm to about 5 cm, 5.5 cm, 6 cm, 6.5 cm, 7 cm, 7.5 cm, or 8 cm. In one example, the length of the housing is 6 cm while the width is 4.5 cm. The volatile composition may vary in color from apparatus to apparatus. The color of the composition may be coordinated with the color of the housing or an indicia on the button to promote a fragrance theme. For instance, the composition may be blue while the indicia on the button, e.g., a hand print, may also be blue to indicate an “ocean” or “calming” theme.

Although not shown in the figures, the first wall may also comprise a second plurality of apertures around the window. The second plurality of apertures may be of equivalent size to one another and may range in number that is two or greater. It should be noted that these apertures are distinct from the primary aperture as well as the window. Without being limited by theory, the second plurality of apertures likely facilitate in the pass through of air in the apparatus thereby increasing the evaporation of the volatile composition and ultimate provision of the composition into the environment.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

The apparatus of the present disclosure is illustrated by the below Examples, which are not to be construed as limitative.

### EXAMPLES

### General Materials and Methods

The Examples below were conducted using Perfume A, which is a mixture comprising: 51% esters; 26% carbonyls; and 15% alcohols, with the balance being composed of various minor components. The components of Perfume A have the following distribution of carbon chain lengths:
16% carbon chain length of from 6 to 8;
50% carbon chain length of from 9 to 11; and
21% carbon chain length of from 12 to 14,
   with the balance being composed of small amounts of other chain lengths.

### Perfume Weight Loss

The following apparatus was used during calculation of the perfume weight loss values detailed in Table 4:
1. Balance (Scale: Ohaus AA210 S/N 11131122540) or equivalent.
2. Housing as depicted in FIGS. 3 and 4.
3. Volatile material cartridges containing 7 ml of perfume composition.
4. 3M Scotch Weld Applicator TC and glue, #3797-TC or equivalent.
5. Evaporation rack or equivalent open tray (baker's) rack, covered at the top and
shelves spaced at 15 cm or more.
6. Room to accommodate evaporating rack with the following measurements, air flow, temperature/relative humidity or equivalent:
   a) Laboratory Dimensions: 32 feet 4 inches long×72 inches wide×108 inches high or 1,730 ft³
   b) Air Flow (Intake and Exhaust)
      Normal Mode: Average Intake Supply: 103.75 ft³/min+6%
      Average Exhaust: 149.25 ft³/min+6%
      Difference results in negative air pressure: −45.5
      Negative pressure indicates that air supply to laboratory and from an adjacent hallway or room is exhausted through the ventilation system.
   c) Temperature and % Relative Humidity
      Average Temperature: 23° C.±0.1° C.
      Average % Relative Humidity: 45%±0.5%

The procedure to determine weight loss is as follows:
1. Load a cartridge with the volatile composition in such a way as to provide a sealed cartridge where the membrane is not yet wetted. For instance, one may pierce a volatile composition cartridge by cutting in it a hole that allows for insertion of an 18 gauge needle.
2. Fill the cartridge with 7 ml of perfume. This is equivalent to 6650 mg of Perfume A, which was used as the standard perfume for all experiments described herein. The volume may need to be adjusted based on the density of the composition of interest.
3. Seal the insertion hole with hot melt adhesive.
4. Measure and record the weight of the apparatus.
5. Insert the cartridge into a housing for holding and orienting the cartridge, and ensure that the cartridge is set correctly within the housing to ensure proper air flow therethrough.
6. Activate the cartridge by any appropriate means to allow contact between the perfume and the membrane, thereby wetting the membrane. In the Examples disclosed herein this activation was achieved by pushing an activation button that breaks a breakable seal between the perfume and the membrane.
7. Record the cartridge weight daily at the same time for a specific time period, e.g. at least sixty days.
8. Determine the weight loss of the volatile composition during the relevant time period.

### Bulk Density

The bulk density of the membrane is determined from the sample weight divided by the sample volume. Sample weight can be measured by a standard weighing balance (e.g. Ohaus AA210 S/N 11131122540 or equivalent). Sample volume can be ascertained from measurements of the sample dimensions using a standard vernier calipers.

### Thickness

The thickness of the membrane can be measured using a standard micrometer screw gauge.

### Porosity

The porosity of the membrane, expressed as percent by volume, is determined according to the following equation:

Porosity=100[1−d1/d2]

where, d1 is the density of the sample, which is determined from the sample weight and the sample volume as ascertained from measurements of the sample dimensions; and d2 is the density of the solid portion of the sample, which is determined from the sample weight and the volume of the solid portion of the sample. The volume of the solid portion of the microporous membrane is determined using a Quantachrome stereopycnometer (Quantachrome Corp.) in accordance with the operating manual accompanying the instrument.

### Volume Average Pore Diameter

The volume average diameter of the pores of the membrane is determined by mercury porosimetry using an Autoscan mercury porosimeter (Quantachrome Corp.) in accordance with the operating manual accompanying the instrument. The volume average pore radius for a single scan is automatically determined by the porosimeter. In operating the porosimeter, a scan is made in the high pressure range (from 138 kilopascals absolute to 227 megapascals absolute). If 2 percent or less of the total intruded volume occurs at the low end (from 138 to 250 kilopascals absolute) of the high pressure range, the volume average pore diameter is taken as twice the volume average pore radius determined by the porosimeter. Otherwise, an additional scan is made in the low pressure range (from 7 to 165 kilopascals absolute) and the volume average pore diameter is calculated according to the equation:

d=2[v₁r₁/w₁+v₂r₂/w₂]/[v₁/w₁+v₂/w₂]

where, d is the volume average pore diameter; v₁ is the total volume of mercury intruded in the high pressure range; v₂ is the total volume of mercury intruded in the low pressure range; r₁ is the volume average pore radius determined from the high pressure scan; r₂ is the volume average pore radius determined from the low pressure scan; w₁ is the weight of the sample subjected to the high pressure scan; and w₂ is the weight of the sample subjected to the low pressure scan.

### Total Pore Volume

The total pore volume of the membrane is determined by mercury porosimetry using an Autoscan mercury porosimeter (Quantachrome Corp.) in accordance with the operating manual accompanying the instrument. The total pore volume for a single scan is automatically determined by the porosimeter.

Where the Examples provided below do not provide results for an Inventive Membrane, this means that the Inventive Membrane in question was not tested in that experiment.

### EXAMPLE 1: PERFUME RELEASE

Inventive Membranes 1-3 were obtained from Microporous, LLC. Comparative Membrane 1 was obtained from PPG Industries, Inc. The properties of the different membranes are provided in Table 3 below.

TABLE 3

| | Comparative | Inventive | Inventive | Inventive |
|---|---|---|---|---|
| Parameter | Membrane 1 | Membrane 1 | Membrane 2 | Membrane 3 |
| Bulk | 0.78 | 0.46 | 0.65 | 0.52 |
| Density | | | | |
| (g/ml) | | | | |
| Total Pore | 0.53 | 1.30 | 0.71 | 0.92 |
| Volume | | | | |
| (ml/g) | | | | |
| Volume | 0.035 | 0.10 | 0.07 | 0.08 |
| Average | | | | |
| Pore | | | | |
| Diameter | | | | |
| (μm) | | | | |
| Porosity | 44 | 59 | 46 | 47 |
| (%) | | | | |
| Thickness | 280 | 250 | 350 | 300 |
| (μm) | | | | |

Two identical air freshening delivery devices as depicted in FIGS. 3 and 4 were prepared using different membranes: one with Inventive Membrane 1, and the other with Comparative Membrane 1. Each device contained 7 ml (6650 mg) of Perfume A housed within a reservoir that, after activation, allowed the perfume to contact the membrane of the device. Each device had 27 cm² of the respective membrane.

The perfume release performance of the air freshening delivery devices was assessed using the “Perfume Weight Loss” procedure outlined above. Results are provided in Table 4 below.

TABLE 4

| | Perfume Weight | Perfume Weight | Increase in |
|---|---|---|---|
| | loss (evaporation) | loss (evaporation) | Evaporation for |
| | for Inventive | for Comparative | Inventive |
| Week | Membrane 1 (mg) | Membrane 1 (mg) | Membrane 1 (mg): |
| 1 | 1233 | 1189 | 44 |
| 2 and 3 | 1958 | 1745 | 213 |
| 4 and 5 | 1485 | 1367 | 120 |
| 6 to 8 | 1001 | 913 | 87 |
| Total | 5677 | 5214 | 464 |
| (1 to 8) | | | |

Each device was loaded with 7 ml (6650 mg) of perfume. Therefore, after eight weeks the device using Inventive Membrane 1 was able to release 85.3% of total perfume, while the device using Comparative Membrane 1 was only able to release 78.4%.

From comparing the results for Inventive Membrane 1 and Comparative Membrane 1, it is clear that Inventive Membrane 1 is able to release more perfume during every stage of the product's lifespan (8 weeks). This improvement is obtained whilst maintaining the same total perfume loading (7 ml) and with the same perfume mixture, indicating that Inventive Membrane 1 improves the efficiency of the perfume release. For the avoidance of doubt, the device prepared using Inventive Membrane 1 retains the same overall product life (around 8 weeks) as that prepared using Comparative Membrane 1.

The final column of Table 4 shows the increase in perfume release at each stage. While the relative increase is lower during week 1, this is because the perfume release at this early stage is primarily driven by the highly volatile top notes (e.g. vapor pressure of at least 0.1 Torr at 25° C.). However, after the first week, when middle and bottom notes contribute a greater amount to perfume evaporation, it is clear that Inventive Membrane 1 drastically outperforms Comparative Membrane 1. This improvement in perfume release results in a lower amount of perfume remaining within the device (whether within the reservoir or on/within the membrane itself) at the end of the product's lifespan (8 weeks), reducing wastage.

While the device prepared using Comparative Membrane 1 has a lifespan of around 8 weeks, a detectable amount of perfume remains within the membrane at end of life, meaning that a consumer is still able to detect a scent and may not realise that the product has reached the end of its life. This leads to consumer dissatisfaction and confusion. In contrast, since Inventive Membrane 1 is able to provide increased evaporation of the perfume, less perfume remains on the membrane at end of life. This results in a less noticeable residual scent, providing a clearer olfactory signal to consumers that the product has reached the end of its life.

### EXAMPLE 2: VISUAL CHANGE

The following apparatus/materials were used during determination of the visual change in the colour and/or transparency of the membrane as detailed in Methods 1 and 3:
1. Balance (Scale: Ohaus AA210 S/N 11131122540) or equivalent.
2. 0.1 ml of perfume composition (Perfume A).
3. Membrane with size of 2.5 cm by 2.5 cm.
4. To obtain membrane after perfume wetting, add 0.1 ml of perfume onto membrane.

The following apparatus/materials were used during determination of the visual change in the colour and/or transparency of the membrane after perfume wetting as detailed in Method 2:
1. Balance (Scale: Ohaus AA210 S/N 11131122540) or equivalent.
2. Housing of the present disclosure including a first and second wall
3. Volatile composition cartridges containing 7 ml of perfume composition (Perfume A, optionally containing a blue dye).
4. 3M Scotch Weld Applicator TC and glue, #3797-TC or equivalent. 5. The procedure to obtain a working device that allows membrane to be wet by perfume is as follows:
   a. Load the cartridge with the volatile composition in such a way as to provide a sealed cartridge that is not yet wetted. For instance, one may pierce a volatile composition cartridge by cutting in it a hole that allows for insertion of an 18 gauge needle.
   b. Fill the cartridge with 7 ml of perfume. This is equivalent to 6650 mg of the standard perfume. The volume may need to be adjusted based on the density of the composition of interest.
   c. Seal the insertion hole with hot melt adhesive.
   d. Insert the cartridge into the housing and ensure that the cartridge is set correctly within the housing to ensure proper air flow therethrough.
   e. Activate the cartridge to wet its membrane. Herein, such activation is achieved by pushing the activation button. There may, however, be equivalent means of activation and wetting the membrane.

The colour/transparency change was assessed by three methods.
Method 1 (Measuring luminous transmittance): Measure luminous transmittance for membrane as-is and after perfume wetting based on ISO 13468-2:2021 Plastics—Determination of the total luminous transmittance of transparent materials—Part 2: Double-beam instrument.
Method 2 (Measuring colour difference of membrane using delta E): Take a picture of the membrane before and after device has been activated. Determine the sRGB values of both pictures using standard software (MS Paint software for Microsoft Windows users, Digital Color Meter for Mac users, https://imagecolorpicker.com/or equivalent), then calculate the Delta E between the pictures based on CIE2000 definition from the International Commission on Illumination (CIE), using standard software (e.g http://colormine.org/delta-e-calculator/cie2000 or https://rgbcmyk.com.ar/en/xla-2/or equivalent).
Method 3 (Panellist test): Conduct a test with 10 panellists by asking them to rate the change in transparency and colour difference of membrane as-is and after perfume addition from a scale of 1 to 5, with 1 being no change, 2 being slight change, 3 being moderate change, 4 being large change and 5 being extreme change.

Results for each method are provided below.

### Method 1

Luminous transmittance for the dry and wet membranes was assessed according to ISO 13468-2:2021. Results are shown in Table 5 below.

TABLE 5

| | Inventive | Inventive | Comparative |
|---|---|---|---|
| | Membrane 1 | Membrane 3 | Membrane 1 |
| Luminous | 2.0 | 7.3 | 2.4 |
| Transmittance (dry)/% | | | |
| Luminous | 49.2 | 57.9 | 13.8 |
| Transmittance (after | | | |
| perfume wetting)/% | | | |
| Change in Luminous | 47.2 | 50.6 | 11.4 |
| Transmittance/% | | | |

The results confirm that Inventive Membranes 1 and 3 display a far greater change in luminous transmittance between the dry and wet states than Comparative Membrane 1.

### Method 2

Delta E was calculated from RGB values as described above for Method 2.

Inventive Membrane 4 corresponds closely to Inventive Membrane 1 except that it also comprises 0.66 wt. % activated charcoal. This resulted in a change from grey when dry to black when wet.

As indicated in Table 6, the perfume formulation used was either colourless or blue.

TABLE 6

| | | | | | | Comparative | |
|---|---|---|---|---|---|---|---|
| | Inventive Membrane | | | | | Membrane | |
| | 1 | 1 | 3 | 3 | 4 | 1 | 1 |
| Coloured | No | Yes | No | Yes | No | No | Yes |
| Perfume | | (blue) | | (blue) | | | (blue) |
| Formulation? | | | | | | | |
| RGB | 218, | 218, | 215, | 215, | 93, 99, | 217, | 217, |
| (membrane | 220, | 220, | 216, | 216, | 102 | 217, | 217, |
| before | 217 | 217 | 211 | 211 | | 206 | 206 |
| device | | | | | | | |
| activation) | | | | | | | |
| RGB | 178, | 89, | 177, | 86, | 0, 2, 0 | 208, | 201, |
| (membrane | 176, | 114, | 173, | 108, | | 206, | 209, |
| after device | 162 | 132 | 155 | 129 | | 192 | 201 |
| activation) | | | | | | | |
| Delta E | 12.2 | 34.6 | 12.4 | 39.9 | 28.9 | 2.9 | 4.2 |

Delta E values below 5 are generally considered to represent the same, or similar, colours (even if a difference is perceptible). Delta E values above 5 are generally considered to represent two different colours (Mokrzycki and Tatol, Machine Graphics and Vision 20(4):383-411):
0<ΔE<1—observer does not notice the difference
1<ΔE<2—only experienced observer can notice the difference
2<ΔE<3:5—unexperienced observer also notices the difference
3:5<ΔE<5—clear difference in colour is noticed
5<ΔE—observer notices two different colours

The results in Table 6 above demonstrate the membranes used in the apparatus of the present disclosure provide a noticeable change of colour when wetted with a perfume (Delta E>5). This applies whether or not the perfume was coloured with a blue dye. In contrast, the comparative membrane did not provide a change of colour (Delta E<5).

For the avoidance of doubt, when a dye is used in the perfume formulation, the blue dye did not penetrate into the membrane during use. This means that the membrane adopts a wetted appearance when in contact with (coloured) perfume, but reverts to its original appearance at end of life. Thus, the Delta E values achieved for Inventive Membranes 1 and 3 with a coloured perfume formulation confirm that Inventive Membranes 1 and 3 are able to provide a strong visual signal that an air freshening device has reached the end of its life. This visual signal is advantageously stronger than that of Comparative Membrane 1.

### Method 3

The average scores from the 10 panellists are provided below. The results show that Inventive Membrane 1 has a much more noticeable change in appearance when in contact with perfume than Comparative Membrane 1. This improvement is clearly detectable with the human eye.

TABLE 7

| | Inventive | Comparative |
|---|---|---|
| | Membrane 1 | Membrane 1 |
| Transparency Change | 3.6 | 1.2 |
| Colour Change | 4.9 | 2.9 |

1 = no change, 2 = slight change, 3 = moderate change, 4 = large change and 5 = extreme change.

The results in Tables 5-7 above demonstrate that there is a drastic appearance, colour and transparency change when Inventive Membranes 1, 3 and 4 are wetted by a volatile material, while the appearance and transparency change for the Comparative Membrane 1 was far less significant.

This confirms that the inventive examples are able to provide an advantageously clear signal that an air freshening apparatus has been correctly activated, and also that it has reached its end of life.

### EXAMPLE 3: SEALING TEMPERATURES

The membrane must be fully sealed to the apparatus to ensure controlled evaporation of the perfume through the membrane and avoid perfume leakage. Sealing may be carried out using a conventional heat-sealing machine at a temperature sufficient to melt the materials of both the membrane and the part of the apparatus to which the membrane will be sealed (e.g. a reservoir for containing perfume). This will then bond the membrane and apparatus together, creating a seal. However, too high a sealing temperature can result in overheating of the surface of the material and induce undesirable transparentization. Higher sealing temperatures are also more energy intensive, increasing commercial production costs.

Therefore, it is desirable to reduce the sealing temperature as much as possible.

The sealing temperatures for the Inventive Membrane 1 and Comparative Membrane 1 are provided in Table 8 below. Inventive Membranes 1 and 2 may be sealed at lower temperatures than Comparative Membrane 1. Inventive Membrane 3 was not tested.

TABLE 8

| | Inventive | Inventive | Comparative |
|---|---|---|---|
| | Membrane 1 | Membrane 2 | Membrane 1 |
| Sealing Temperature | 180-190 | 180-190 | 200-220 |
| (° C.) | | | |

The above Examples demonstrate the following benefits provided by an apparatus utilising a microporous membrane as defined herein.
1) The membrane enables a greater efficiency of use of perfume provided in the apparatus, with less volatile material left trapped on the membrane at the end of the product's lifespan.
2) The membrane enables improved perfume release, especially during the middle and end portions of the product's lifespan. This benefit is surprisingly obtained whilst maintaining the same total product lifespan.
3) The membrane has a substantially different appearance when wetted with volatile material, as compared to its appearance when dry (whether before activation or at the end of the product's lifespan). This advantageously allows a consumer to easily determine whether or not the product has been activated properly, and also whether it needs replacing. This provides a two-fold benefit to consumer satisfaction.
   a. The first benefit arises because consumers may be dissatisfied when an apparatus as described in U.S. Pat. No. 8,740,110 or US 2022/0047754 is activated, since they will notice the amount of volatile material in the reservoir decreasing rapidly immediately after activation as it passes through the rupturable substrate, but there is no clear signal that the volatile material is contacting the membrane.
   b. The second benefit arises because consumers may more easily determine that the product has reached the end of its life.
4) The membrane, having a lower bulk density to prior art membranes, may be sealed to the apparatus at a lower temperature, improving ease and cost of manufacture.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any example disclosed or claimed herein or that it alone, or in any combination with any other reference or references, teaches, suggests or discloses any such example. Further, to the extent that any meaning or definition of a term in this document conflicts with any meaning or definition of the same term in a document incorporated by reference, the meaning or definition assigned to that term in this document shall govern.

While particular examples of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. An apparatus for delivering a volatile material comprising a delivery engine comprising:
a reservoir containing a volatile material; and
a microporous membrane enclosing said reservoir, wherein:
the microporous membrane has a volume average pore diameter of from 0.065 μm to 0.15 μm.

2. The apparatus of claim 1, wherein:
the delivery engine further comprises:
a rupturable substrate secured to said reservoir; and
a rupture element positioned adjacent to said rupturable substrate, and
the microporous membrane encloses said rupturable substrate and said rupture element.

3. The apparatus of claim 1, wherein the microporous membrane has a surface area of from 2 cm² to 100 cm².

4. The apparatus of claim 1, wherein the microporous membrane has a porosity of from 45 to 70%.

5. The apparatus of claim 1, wherein the microporous membrane has a total pore volume of from 0.6 to 2 cm³/g.

6. The apparatus of claim 1, wherein the microporous membrane has a bulk density of from 0.3 to 0.8 g/cm³.

7. The apparatus of claim 1, wherein the microporous membrane has a thickness of from 0.2 to 0.4 mm.

8. The apparatus of claim 1, wherein the microporous membrane has:
a porosity of from 45 to 60%;
a total pore volume of from 0.65 to 1.5 cm³/g; and
a bulk density of from 0.35 to 0.75 g/cm³.

9. The apparatus of claim 1, wherein the microporous membrane has:
a surface area of from 2 cm² to 100 cm²;
a porosity of from 45 to 70%;
a total pore volume of from 0.65 to 1.6 cm³/g;
a bulk density of from 0.35 to 0.75 g/cm³; and
a thickness of from 0.22 to 0.37 mm.

10. The apparatus of claim 1, wherein the microporous membrane has:
a surface area of from 2 cm² to 35 cm²;
a porosity of from 45 to 60%;
a total pore volume of from 0.7 to 1.5 cm³/g;
a bulk density of from 0.4 to 0.7 g/cm³; and
a thickness of from 0.25 to 0.35 mm.

11. The apparatus of claim 1, wherein the microporous membrane is not laminated.

12. The apparatus of claim 1, wherein the microporous membrane comprises polyethylene.

13. The apparatus of claim 12, wherein the polyethylene comprises ultra-high molecular weight polyethylene (UHMWPE).

14. The apparatus of claim 1, wherein the microporous membrane:
comprises polyethylene;
has a thickness of from 0.2 to 0.4 mm; and
is not laminated.

15. The apparatus of claim 1, wherein:
the reservoir contains a volatile material; and
the volatile material is liquid at 25° C.

16. The apparatus of claim 15, wherein the volatile material has a vapor pressure of at least 8 Pa at 25° C.

17. The apparatus of claim 15, wherein the volatile material has a vapor pressure of at least 30 Pa at 25° C.

18. The apparatus of claim 1, wherein:
the delivery engine further comprises:
a rupturable substrate secured to said reservoir, and
a rupture element positioned adjacent to said rupturable substrate;
the microporous membrane encloses said rupturable substrate and said rupture element;
the reservoir contains a volatile material;
the apparatus is configured such that activation of the rupture element allows contact between the volatile material and the microporous membrane; and
the apparatus is configured such that after activation of the rupture element, the apparatus releases at least 80 wt. % of the volatile material within a time period of 8 weeks at a temperature of 25° C.

19. The apparatus of claim 1, wherein the microporous membrane has a first visible state when dry, and a second visible state when wetted with volatile material, and where a CIE2000 Delta E value between the first visible state and second visible state is greater than or equal to 5.

20. The apparatus of claim 1, wherein the microporous membrane has a first visible state when dry, and a second visible state when wetted with volatile material, and where a difference between a luminous transmittance value for the first visible state and a luminous transmittance value for the second visible state, as measured by ISO 13468-2:2021, is greater than or equal to 25%.
